(19) United States
(12) Patent Application Publication
(10) Pub. No.: US 2009/0255536 A1
(43) Pub. Date: Oct. 15, 2009

(54) Biofilm-Inhibiting Catheters and Tubings

(75) Inventors: Yossef Av-Gay, Vancouver (CA); Livia Mahler, Vancouver (CA); Christopher C. Miller, North Vancouver (CA)

Correspondence Address: FULBRIGHT & JAWORSKI L.L.P., 600 CONGRESS AVE., SUITE 2400, AUSTIN, TX, 78701, US

(73) Assignee: ENOX BIOPHARMA, INC., Vancouver (CA)

(21) Appl. No.: 12/420,671

(22) Filed: Apr. 8, 2009

Related U.S. Application Data
(60) Provisional application No. 61/043,639, filed on Apr. 9, 2008.

(51) Int. Cl. A61L 29/16; A61M 16/04; A61M 27/00; B05D 7/24

(52) U.S. Cl. 128/207.14; 604/265; 604/543; 604/544; 427/2.25

(57) ABSTRACT

Antimicrobial and biofilm-inhibiting nitric oxide-releasing medical appliances for installation into a patient, and processes for producing the medical appliances. The medical appliances are configured for conveying materials into and out of the patient's body and may comprise tubings or pouching systems. The medical appliances comprise a gas-permeable cured resin material selected from the group consisting of curable silicones, polyvinyl acetates, thermoplastic elastomers, acrylonitrile-butadiene-styrene copolymer rubber, polyurethanes and selected combinations thereof, wherein the gas-permeable cured resin material comprises a matrix suitable for releasably sequestering therein permeating gases. A plurality of nitric-oxide gas-releasing moieties is sequestered within and through the gas-permeable resin material. Nitric oxide is released from at least a portion of said plurality of nitric oxide gas-releasing moieties upon contact of the medical appliance with a moisture source. Exemplary medical appliances include endotracheal tubes, crichotracheotomy tubes, urinary catheters, wound drainage tubes, central intravenous catheters, peripheral intravenous catheters, and poucing systems.

### CROSS REFERENCE TO RELATED APPLICATIONS

This application claims priority to U.S. Provisional Application No. 61/043,639 filed Apr. 9, 2008, the entire contents of which are specifically incorporated by reference herein without disclaimer.

### BACKGROUND OF THE INVENTION

1. Field of the Invention

This invention relates to medical appliances and devices. More particularly, this invention relates to medical appliances and devices comprising antimicrobial and/or biofilm-inhibiting components.

2. Description of Related Art

Numerous types of medical tubings are provided for delivery of stabilizing treatments to patients during surgical procedures, for post-surgical recovery and/or for periods of convalescence. For example, endotracheal tubes are inserted into a patient's trachea to a point approximately two centimetres above the bifurcation of the lungs, to ensure that air is able to reach the patient's lungs during surgery, emergency medical procedures, intensive care, and mechanical ventilation. Foley catheters are commonly used for passive drainage of a patient's urine from their bladder during periods of convalescence. Foley catheters are flexible tubes that are passed into the patient's bladder through their urethra and are retained therein by a balloon at the tip of the catheter. The balloon is inflated with sterile water after the Foley catheter is installed. Council tip catheters are modified Foley catheters provided with three channels wherein the first is the primary drainage channel, the second is a narrow channel configured introducing an irrigant into the patient's bladder to wash away post-surgery wound debris, while the third channel is connected with the retaining balloon for the introduction or removal of sterile water for inflation or deflation the retaining balloon as post-surgery healing proceeds. Wound drainage tubes are often installed during completion of many surgical procedures for draining-off bodily fluids that collect at operation sites. Wound drainage tubes may be hooked to a suction device or alternatively, may be left to drain by gravity. Depending on the type of surgery and/or the amount of drainage, a patient may have a wound drainage tube installed for a period ranging from one day to several weeks. Exemplary wound drainage tubes include Jackson-Pratt® drains (Jackson-Pratt is a registered trademark of Allegiance Corp., McGraw Park, Ill., USA) and Penrose drains.

The human body surface is populated by a wide diversity of microorganisms which in healthy individuals, are considered to be normal populations of commensal microorganisms that typically do not cause any clinical problems for the individuals. However, serious microbial infections of trauma or surgical wound sites can result if certain microorganisms commonly resident on human body surfaces, gain access to bodily fluids associated with and/or released from the wound sites. Patients maintained and recuperating in medical care facilities are also at risk of opportunistic infections by antibiotic-resistant microbial strains associated with clinical environments, adding to the risk of infection from the patient's own microbial flora. Complications resulting from wound site infections include delay and perturbation of wound healing, development of more serious secondary microbial infections at the wound sites, and spreading to other parts of the patient's body via their vascular system (i.e., septicemia).

Body surface incision sites for the insertion of cricotracheotomy tubes and intravenous tubes for venal delivery of nutrient solutions or blood, after installation of the tubes are nutrient-rich staging sites for infections by opportunistic bacteria, as are entry-point sites for endotracheal tubes and Foley urinary catheters. Catheter-acquired urinary track infections (CAUTI) commonly occur in hospital environments as a result of localized wound infections (superficial or deep-sided), caused by insertion of bladder catheters for urine drainage. Trauma associated with endotracheal tube installation may result in the onset of pneumonia which, in combination with impaired breathing/coughing as a result of sedation or analgesics during the first few hours of recovery after trauma and/or surgery, may endanger the health of patients after surgery. Infections can also result from the post-installation proliferation of opportunistic pathogens along the surfaces of endotrachial tubes and of gastrointestinal tubes. Such medical complications are typically the consequence of: (a) the formation of biofilms across the outer and inner surfaces of the installed catheters or tubes from which certain nosocomial opportunistic microbial members of the biofilm microbial community colonize and penetrate the patient's wound tissue, and (b) microbial infections of the body's fluid conveyance pathways, e.g., their vascular systems and interconnections between their organs.

Certain medical conditions require individuals to undergo temporary or permanent colostomy procedures which generally comprise connecting a part of their colon onto their anterior abdominal wall leaving an opening on the abdomen called a stoma. The stoma is formed from the end of the large intestine which is drawn out through the incision and sutured to the skin. After a colostomy, fecal matter produced during digestion leaves the patient's body through the stoma into sealed collection system commonly called an “ostomy pouching system”. Ostomy pouching systems usually consist of a mounting plate (also referred to as a “wafer”) and a collection pouch that is mechanically attached in the mounting plate with an airtight seal. The ostomy mounting plate is configured for sealable attachment around the individual's stoma such that there is minimal contact of the fecal matter with the skin immediately adjacent the stoma whereby localized infections may occur. One-piece ostomy pouching systems are designed for a single-use attachment to an individual's body. Two-piece ostomy pouching systems are designed for the mounting plate to be attached to an individual's body for several days while multiple pouches are attached to and then detached from the mounting plate.

Formation of a biofilm on an installed catheter or tube or ostomy pouching system mounting plate begins with the attachment of free-floating planctonic microorganisms to the outer or inner surface of the tube. These first colonists adhere to the surface initially through weak, reversible forces and if not immediately separated or repelled from the surface, they can anchor themselves more permanently using cell adhesion organelles such as pili and molecules such as polysaccharides, lipopolysaccharides and proteins. The first colonists facilitate the arrival of other cells by providing more diverse adhesion sites and beginning to build the matrix that holds the biofilm together. Some species are not able to attach to a surface on their own but are often able to anchor themselves to the matrix or directly to earlier colonists. Once colonization has begun, the biofilm grows through a combination of cell division and recruitment. The final stage of biofilm formation is known as development, and is the stage in which the biofilm is established and may only change in shape and size. This development of biofilm results in a complex aggregation of microorganisms characterized by the excretion of a protective and adhesive matrix that allows the cells as an aggregate. Bacteria living in biofilms usually have significantly different properties from free-floating planktonic bacteria of the same species, as the dense and protected environment of the film allows them to cooperate and interact in various ways. One benefit of this environment is increased resistance to detergents and antibiotics, as the dense extracellular matrix and the outer layer of cells protect the interior of the community. Consequently, microorganisms resident in biofilms become significantly more antibiotic resistant.

Current medical strategies in use for preventing biofilm development on installed medical tubings and catheters and subsequent microbial infection of the patient's wound tissues and physiological systems are primarily preventative, focusing on the use of sterile techniques during surgery and post-trauma tubing installations, and the topical and oral administration of antimicrobial therapies (i.e., antimicrobial prophylaxis. However, even when sterile techniques are carefully adhered to, surgical procedures can introduce bacteria resulting in infections caused by opportunistic bacteria present in clinical environments. Therefore, antibiotic prophylaxis strategies are carefully developed and administered for many surgical and trauma treatment procedures.

### SUMMARY OF THE INVENTION

The exemplary embodiments of the present invention, are directed to antimicrobial and biofilm-inhibiting nitric oxide-releasing medical tubings and pouching systems configured for installation into a patient's body for sustaining bodily functions. The NO-releasing medical tubings and NO-releasing pouching systems are also suitable for delivery of medical treatments during surgeries, emergency medical procedures, post-surgical or emergency care recovery periods, and during long-term convalescence. Suitable medical tubings are exemplified by urinary catheters, central venous catheters, peripheral venous catheters, endotracheal tubes, cricotracheotomy tubes and the like, and will be generally referred to from herein as “medical tubings”. Suitable pouching systems are exemplified by ostomy pouching systems and the like.

According to one aspect, the cured gas-permeable resin material comprises curable silicones.

According to another aspect, the antimicrobial/biofilm-inhibiting characteristics are provided by nitric oxide molecules.

According to another aspect, the antimicrobial/biofilm-inhibiting nitric oxide-releasing molecules are exemplified by nitric oxide molecules.

According to another aspect, the antimicrobial/biofilm-inhibiting nitric oxide-releasing molecules are exemplified by compositions configured to controllably release nitric oxide molecules upon contact with moisture exemplified by body fluids and fluid therapeutic compositions for administration to a patient. Exemplary body fluids are exemplified by blood, urine, mucus and saliva.

According to a further aspect, the antimicrobial/biofilm-inhibiting nitric oxide-releasing medical tubings are exemplified by endotracheal tubes and crichotracheotomy tubes, urinary catheters, central venous catheters and peripheral catheters, wound drainage tubings, and the like.

According to another exemplary embodiment, the antimicrobial/biofilm-inhibiting nitric oxide-releasing pouching system is exemplified by the mounting plates of ostomy pouching systems.

According to another exemplary embodiment of the present invention, the antimicrobial/biofilm-inhibiting gas-releasing medical tubings are produced with a process whereby fully configured and cured gas-permeable resin-based tubes are controllably saturated with a selected antimicrobial/biofilm-inhibiting gas exemplified by nitric oxide (e.g., gNO), whereby the resin-based tubes releasably sequester antimicrobial gas molecules. The antimicrobial/biofilm-inhibiting gNO-saturated medical tubings are individually packagable in gas-impermeable containers.

According to a further embodiment, the antimicrobial/biofilm-inhibiting gas-releasing medical tubings are produced by intermixing a suitable selected chelating agent saturated with antimicrobial gas molecules, with a curable polymeric resin material. The intermixed material is formed and configured into a plurality of antimicrobial gas-releasing medical tubings, which are then cured. After curing, the antimicrobial gas-releasing medical tubings are individually packagable and sealed into gas-impermeable containers.

According to a further embodiment, the antimicrobial/biofilm-inhibiting gas-releasing medical tubings are produced by coating a curable polymeric resin material with a chemical composition configured to release nitric oxide, which is then cured. After curing, the antimicrobial gas-releasing medical tubings are individually packagable and sealed into gas-impermeable containers.

According to one aspect, the chemical composition is configured to release nitric oxide upon contact with a bodily fluid exemplified by blood, urine, mucus and saliva.

### BRIEF DESCRIPTION OF THE DRAWINGS

The present invention will be described in conjunction with reference to the following drawing, in which:

FIG. 1 is a chart showing the total accumulation of nitrites produced from catheters impregnated with nitric oxide according to an exemplary embodiment of the present invention;

FIG. 2(A) is a chart showing the release of nitrites from nitric oxide-impregnated catheters during a 14-day period after the catheters were immersed in water, 2(B) is a chart showing the production of nitrites as a direct correlation of nitric oxide release during a 14-day period from catheters that had been stored for 7 days after impregnation with nitric oxide and then immersed in water, and 2(C) is a chart showing the release of nitrites from nitric oxide-impregnated catheters during a 14-day period while immersed in sterile urine (sterile urine changed daily);

FIG. 3(A) is a photograph comparing colonization of Escherichia coli on control catheters and on nitric oxide-impregnated catheters after immersion of the catheters for 24 h in a suspension comprising 10² CFU mL⁻¹ of E. coli, 3(B) is a photograph comparing colonization of E. coli on control catheters and on nitric oxide-impregnated catheters after immersion of the catheters for 24 h in a suspension comprising 10³ CFU mL⁻¹ of E. coli, 3(C) is a photograph comparing colonization of E. coli on control catheters and on nitric oxide-impregnated catheters after immersion of the catheters for 24 h in a suspension comprising 10⁴ CFU mL⁻¹ of E. coli. Each of the photographs is a 3-compartment Petri plate where a selected immersed catheter was rolled over the surface of one compartment;

FIG. 4(A) is a photograph comparing growth of E. coli on LB agar following isolations from control catheters and from nitric oxide-impregnated catheters that had been immersed for 24 h in suspensions comprising 10² CFU mL⁻¹ of E. coli, 4(B) is a photograph comparing growth of E. coli on LB agar following isolations from control catheters and from nitric oxide-impregnated catheters that had been immersed for 24 h in suspensions comprising 10³ CFU mL⁻¹ of E. coli, 4(C) is a photograph comparing growth of E. coli on LB agar following isolations from control catheters and from nitric oxide-impregnated catheters that had been immersed for 24 h in a suspension comprising 10⁴ CFU mL⁻¹ of E. coli. Each Petri plates in the photographs is a 3-compartment Petri plate wherein each compartment was surface-plated with an aliquot of an E. coli suspension wherein a selected catheter had been previously immersed;

FIG. 5 is a chart comparing numbers of E. coli from FIG. 4 before and after the 24-h incubation of catheters in the E. coli suspensions. The black bars are data from the control catheters, while the white bars are data from the nitric oxide-impregnated catheters;

FIG. 6(A) is a photograph comparing colonization of E. coli on control catheters and on nitric oxide-impregnated catheters, after both sets of catheters had been stored for 7 days in a sterile air environment, after post-storage immersion of the catheters for 1 min in a suspension comprising 10² CFU mL⁻¹ of E. coli, followed by a 24-h incubation in PBS after which, a selected catheter was rolled onto the surface of a compartment in a selected Petri plate, and 6(B) is a photograph comparing colonization of E. coli on control catheters and on nitric oxide-impregnated catheters, after both sets of catheters had been stored for 7 days in sterile water, after post-storage immersion of the catheters for 1 min in a suspension comprising 10² CFU mL⁻¹ of E. coli, followed by a 24-h incubation in PBS after which, a selected catheter was rolled onto the surface of a compartment in a selected Petri plate; and

FIG. 7(A) is a photograph comparing colonization of E. coli on control catheters and on nitric oxide-impregnated catheters, after both sets of catheters had been stored for 7 days in a sterile air environment, after post-storage immersion of the catheters for 1 min in a suspension comprising 10² CFU mL⁻¹ of E. coli, followed by a 24-h incubation in PBS, and 7(B) is a photograph comparing colonization of E. coli on control catheters and on nitric oxide-impregnated catheters, after both sets of catheters had been stored for 7 days in sterile water, after post-storage immersion of the catheters for 1 min in a suspension comprising 10² CFU mL⁻¹ of E. coli, followed by a 24-h incubation in PBS. Each Petri plates in the photographs is a 3-compartment Petri plate wherein each compartment was surface-plated with an aliquot of suspension from a selected catheter previously immersed in a selected E. coli suspension; and

FIG. 8 is a chart comparing growth of E. coli on LB agar following isolations from 10³ CFU mL⁻¹ E. coli suspensions wherein control catheters and from nitric oxide-impregnated catheters that had been previously stored immersed in sterile water for 12 days, were immersed for 24 h. The horizontal-striped bars show the control catheter data, while the checkered bars show the nitric oxide-impregnated catheter data.

### DETAILED DESCRIPTION OF THE INVENTION

The exemplary embodiments of the present invention are directed to self-sterilizing NO-releasing biofilm-inhibiting antimicrobial medical appliances and devices configured for installation into a patient's body for sustaining bodily functions and/or delivery of medical treatments during surgeries, emergency medical procedures, post-surgical or emergency care recovery periods, and during long-term convalescence. Certain self-sterilizing NO-releasing biofilm-inhibiting antimicrobial medical appliances of the present invention are exemplified by urinary catheters, central venous catheters, peripheral venous catheters, endotracheal tubes, cricotracheotomy tubes and the like, and will be generally referred to from hereon in as “medical tubings”. The biofilm-inhibiting nitric oxide-releasing antimicrobial medical appliances of the present invention are also exemplified by mounting plates of one-piece and two-piece ostomy pouching systems. The biofilm-inhibiting medical appliances according to the present invention generally comprise materials that are controllably permeatable with gases selected for their antimicrobial properties. Other biofilm-inhibiting NO-releasing antimicrobial medical appliances of the present invention are exemplified by ostomy pouching systems and in particular, the mounting plates of ostomy pouching systems.

The biofilm-inhibiting antimicrobial medical tubings of the present invention are characterized by their biological compatibility with biological tissues associated with internal surfaces of passageways into and within a patient's body, e.g., throats, urethras and veins. The antimicrobial medical tubings generally comprise polymeric materials exemplified by resins which after forming and curing, are microporous and have suitable high gas permeability and/or sequestering properties needed to prepare the antimicrobial medical tubings of the present invention. These resins are suitably characterized by their ability for sequestering selected antimicrobial gases that have intercalated therein, and then, after contact with moisture, releasing the antimicrobial gases over extended periods of time. Suitable resins are exemplified by curable silicones, polyvinyl acetates, thermoplastic elastomers, acrylonitrile-butadiene-styrene copolymer rubber, polyurethanes and the like. Curable silicone resins are particularly suitable for the manufacture of the antimicrobial medical tubings of the present invention due to their molecular structure which provides good flexibility both microscopically and macroscopically, and high gas permeability rates. Table 1 illustrates the gas permeability of silicone resins in comparison with other types of materials suitable for such tubular manufacture.

TABLE 1
Gas permeation through selected materials (cc/0.001 in/100.0
in²/24 h at 22.8° C., 0% relative humidity, ASTM D-1434)*.

Permeating gas

| Tubular material | O₂ | CO₂ |
|---|---|---|
| Silicone | 50,000 | 300,000 |
| Urethanes | 200 | 3,000 |
| Epoxies | 5-10 | 8 |
| Fluorocarbons | 7-15 | 15-30 |
| Nylon | 2.6 | 4.7 |
| Polybutylene | 385 | 825 |
| Polycarbonate | 258 | 775 |
| Cellulose acetate | 23 | 105 |

*adapted from; (1) Packaging Encyclopedia 1988 Vol. 33 No. 5, pp. 54-55, and Machine Design, May 25, 1967, p. 192.

The geometries of the antimicrobial medical tubings are generally cylindrical and may simply comprise elongate hollow conduits having the same diameter extending from end to end, or alternatively, may comprise elaborate configurations that may additionally include abrupt diameter changes and odd shaped flanges, and additionally, may be provided with inflatable balloons at their distal ends.

Gaseous nitric oxide (gNO) is an intermediary compound produced during the normal functioning of numerous biochemical pathways in many biological systems including humans. gNO is known to those skilled in these arts as a key biological messenger signaling compound that plays key roles in many biological processes. Recent evidence (e.g., Ghaffari et al., 2005 Nitric Oxide 14: 21-29) suggests that gNO plays an important role in mammalian host defence against infection and regulates wound healing and angiogenesis. In particular, topical applications of exogenous gNO at 200 ppm for extended periods of time inhibited and prevented the growth of a wide range of microbial pathogens Staphylococcus aureus, Escherichia coli, Group B Streptococcus, Pseudomonas aeruginosa, and Candida albicans, without any cytotoxic effects on cultured human dermal fibroblasts. Furthermore, McMullin et al. (2005, Respir. Care 5:1451-1456) demonstrated that exogenous gNO at a concentration of 200 ppm could clear nosocomial pneumonia caused by microbial pathogens such as S. aureus and P. aeruginosa, in about 6 hours. Furthermore, gNO is a vasodilator and therefore, its release about a wound site will result in migration of a patient's white blood cells to the wound site thereby enhancing the patient's innate immune responses. Accordingly, gNO is a particularly suitable antimicrobial gas for saturatingly permeating medical tubings comprising gas-permeable polymeric materials.

The exemplary antimicrobial medical tubings of the present invention are produced by first casting a desired tubular configuration with a selected suitable resin using conventional methods known to those skilled in these arts. It is suitable to process the tubes into their final configuration and finish after which, the tubes are placed into a sealable chamber. The chamber is then saturated with a selected antimicrobial gas, exemplified by gNO, for a selected period of time suitable for infiltratingly saturating the medical tubings whereby the gas is sequestered into and within the resin structure comprising the tubes thereby by providing antimicrobial properties to the medical tubings. It is within the scope of the present invention to provide a deliver gNO from the range of about 5 ppm to about 1,000,000 ppm at a flow rate ranging from about 1 cc mL⁻¹ to about 1,000 cc mL⁻¹ for a period of time ranging from about 1 min to about 120 h. A suitable saturation method comprises delivery of about 500 ppm to about 100,000 ppm gNO at a flow rate ranging from about 5 cc mL⁻¹ to about 100 cc mL⁻¹ for a period of time ranging from about 30 min to about 48 h. Another suitable saturation method comprises delivery of about 5,000 ppm to about 50,000 ppm gNO at a flow rate of about 10 cc mL⁻¹ to about 75 cc mL⁻¹ for a period of time ranging from about 1 h to about 36 h. Another suitable saturation method comprises delivery of about 10,000 ppm to about 35,000 ppm gNO at a flow rate of about 15 cc mL⁻¹ to about 40 cc mL⁻¹ for a period of time ranging from about 6 h to about 30 h. Another suitable saturation method comprises delivery of about 15,000 ppm to about 30,000 ppm gNO at a flow rate of about 20 cc mL⁻¹ to about 35 cc mL⁻¹ for a period of time ranging from about 12 h to about 30 h. Another suitable saturation method comprises delivery of about 18,000 ppm to about 25,000 ppm gNO at a flow rate of about 25 cc mL⁻¹ to about 35 cc mL⁻¹ for a period of time ranging from about 18 h to about 30h. Another suitable saturation method comprises delivery of about 20,000 ppm gNO at a flow rate of about 30 cc mL⁻¹ for a period of time of about 24 h. Excess gNO is then evacuated from the chamber after which, the gNO-loaded medical tubings are removed and individually packaged into gas-impermeable containers. It is optional to impregnate the tubings or other medical appliances with gNO prior to packaging. It is within the scope of the present invention to infiltrate the chamber with a semi-porous sealing gaseous material configured to at least partially cross-link with the outer surfaces of the antimicrobial medical tubings thereby enabling a further extension of time duration for release of the sequestered gas about the antimicrobial medical tubings. The chamber may be controllably infiltrated with the semi-porous sealing gaseous material concurrently with evacuation of the antimicrobial gas from the chamber or alternatively, the antimicrobial gas may be completely evacuated from the chamber after which, the semi-porous sealing gaseous material may be infiltrated into the chamber. Excess semi-porous sealing gaseous material is then evacuated from the chamber after which, the gNO-loaded medical tubings are removed and individually packaged into gas-impermeable containers. Alternatively, it is within the scope of the present invention to impregnate spools of medical tubings with gNO prior to producing and processing tubes into their final configurations.

Other exemplary antimicrobial medical appliances and devices of the present invention exemplified by pouching systems and by mounting plates of ostomy pouching systems, are produced by first casting a desired configuration with a selected suitable material using conventional methods known to those skilled in these arts. It is suitable to process the appliances into their final configuration and finish after which, the appliances are placed into a sealable chamber. The chamber is then saturated with a selected antimicrobial gas, exemplified by gNO, for a selected period of time suitable for infiltratingly saturating the medical appliances whereby the gas is sequestered into and within the resin structure comprising the appliances thereby by providing antimicrobial properties to the medical appliances. It is within the scope of the present invention to provide a deliver gNO from the range of about 5 ppm to about 1,00,000 ppm at a flow rate ranging from about 1 cc mL⁻¹ to about 200 cc mL⁻¹ for a period of time ranging from about 15 min to about 120 h. A suitable saturation method comprises delivery of about 500 ppm to about 100,000 ppm gNO at a flow rate ranging from about 5 cc mL⁻¹ to about 100 cc mL⁻¹ for a period of time ranging from about 30 mm to about 48 h. Another suitable saturation method comprises delivery of about 5,000 ppm to about 50,000 ppm gNO at a flow rate of about 10 cc mL⁻¹ to about 75 cc mL⁻¹ for a period of time ranging from about 1 h to about 36 h. Another suitable saturation method comprises delivery of about 10,000 ppm to about 35,000 ppm gNO at a flow rate of about 15 cc mL⁻¹ to about 40 cc mL⁻¹ for a period of time ranging from about 6 h to about 30 h. Another suitable saturation method comprises delivery of about 15,000 ppm to about 30,000 ppm gNO at a flow rate of about 20 cc mL⁻¹ to about 35 cc mL⁻¹ for a period of time ranging from about 12 h to about 30 h. Another suitable saturation method comprises delivery of about 18,000 ppm to about 25,000 ppm gNO at a flow rate of about 25 cc mL⁻¹ to about 35 cc mL⁻¹ for a period of time ranging from about 18 h to about 30 h. Another suitable saturation method comprises delivery of about 20,000 ppm gNO at a flow rate of about 30 cc mL⁻¹ for a period of time of about 24 h. Excess gNO is then evacuated from the chamber after which, the gNO-loaded medical appliances are removed and individually packaged into gas-impermeable containers. It is within the scope of the present invention to infiltrate the chamber with a semi-porous sealing gaseous material configured to at least partially cross-link with the outer surfaces of the antimicrobial medical appliances thereby enabling a further extension of time duration for release of the sequestered gas about the antimicrobial medical appliances. The chamber may be controllably infiltrated with the semi-porous sealing gaseous material concurrently with evacuation of the antimicrobial gas from the chamber or alternatively, the antimicrobial gas may be completely evacuated from the chamber after which, the semi-porous sealing gaseous material may be infiltrated into the chamber. Excess semi-porous sealing gaseous material is then evacuated from the chamber after which, the gNO-loaded medical appliances are removed and individually packaged into gas-impermeable containers.

It is also within the scope of the present invention to incorporate gNO-sequestering chelating agents into a suitable selected resin material prior to forming medical tubings and/or the medical appliances of the present invention. Alternatively, it is also suitable to coat the surfaces of medical tubings and/or appliances with gNO-sequestering chelating agents. Suitable gNO-sequestering chelating agents are exemplified by sodium nitrite, nitrosothiols, dipyridoxyl chelating agents, L-arginine, organic nitrates, organic nitrites, thionitrates, thionitrites, N-oxo-N-nitrosamines, N-nitrosamines, sydnonimines, 2-hydroxyimino-5-nitro-alkenamides, diazenium diolates, oxatriazolium compounds, oximes, syndomines, molsidomine and derivatives thereof, pirsidomine, furoxanes, nitrosonium salts, and the like, and combinations thereof. A suitable amount of a selected gNO-sequestering chelating agent is placed into a sealable chamber which is then saturated with gNO. A suitable amount of the gNO-loaded chelating agent is then thoroughly intermixed and commingled with a selected resin material after which, the resin material is processed into medical tubings or appliances using methods known to those skilled in these arts. The medical tubings and medical appliances comprising interspersed therethrough gNO-loaded chelating agent, are then sealably packaged into gas-impermeable containers.

An exemplary antimicrobial gas-permeated endotracheal tube of the present invention can be installed through a patient's mouth into their throat and then into their trachea using well-known procedures so that the distal end of the endotracheal tube is about two centimeters from the bifurcation of the lungs while the proximal end of the endotracheal tube extends from the patient's mouth for connection to a suitable mechanical ventilator. The antimicrobial gas sequestered within the resin material comprising the inserted endotracheal tube will slowly diffuse from and about the tube thereby preventing the formation of biofilms thereon the outer and inner surfaces of the tube, while concurrently alleviating and/or preventing post-operative microbial infections normally associated with these types of tubes and without adverse toxicology reactions exemplified by irritation and inflammation, of the mouth, throat and tracheal tissues.

An exemplary embodiment of a biofilm-inhibiting antimicrobial medical tubing comprises a tubing impregnated with nitric oxide, and additionally coated on its outer surface with a chemical composition comprising at least one gNO-sequestering chelating agent exemplified by sodium nitrite, nitrosothiols, dipyridoxyl chelating agents, L-arginine, organic nitrates, organic nitrites, thionitrates, thionitrites, N-oxo-N-nitrosamines, N-nitrosamines, sydnonimines, 2-hydroxyimino-5-nitro-alkenamides, diazenium diolates, oxatriazolium compounds, oximes, syndomines, molsidomine and derivatives thereof, pirsidomine, furoxanes, nitrosonium salts, and the like, and combinations thereof. The nitric oxide-impregnated tubing coated- with a gNO-sequestering chemical composition may optionally additionally, or alternatively, be coated with a chemical composition configured to react with one or more constituents of bodily fluids to produce nitric oxide therefrom.

An exemplary antimicrobial gas-permeated urinary catheter of the present invention can be installed through a patient's urethra into their bladder and then set in place using well-known procedures so that the balloon at distal end of the catheter is expanded to hold the catheter in place, while the proximal end of the urinary catheter extends out from the patient's urethra for connection to a suitable urine drainage bag. The antimicrobial gas sequestered within the resin material comprising the inserted urinary catheter will slowly diffuse from and about the catheter thereby preventing the formation of biofilms thereon the outer and inner surfaces of the catheter, while concurrently alleviating and/or preventing post-operative catheter-acquired urinary track infections normally associated with these types of catheters and without adverse toxicology reactions exemplified by irritation and inflammation, of the urethra and bladder tissues. It is also within the scope of the present invention to impregnate the balloon components cooperable with such catheters, with gNO so that the components release NO upon contact with moisture.

An exemplary antimicrobial gas-permeated central venous catheter of the present invention can be installed into a patient's selected large vein in the neck, chest or groin so that the distal end of the catheter extends a suitable length into the vein, while the proximal end of the central venous catheter extends out from the patient's body for connection to an IV bag containing a selected intravenous fluid or drug. The antimicrobial gas sequestered within the resin material comprising the inserted central venous catheter will slowly diffuse from and about the catheter thereby preventing the formation of biofilms thereon the outer and inner surfaces of the catheter, while concurrently alleviating and/or preventing post-operative microbial infections normally associated with these types of catheters and without adverse toxicology reactions exemplified by irritation and inflammation, of the patient's body tissues the catheter is installed therethrough. Similarly, an exemplary antimicrobial gas-permeated peripheral venous catheter of the present invention can be installed into a patient's vein in their arm or wrist for intravenous delivery of selected fluids and/or drugs.

### EXAMPLES

### Example 1

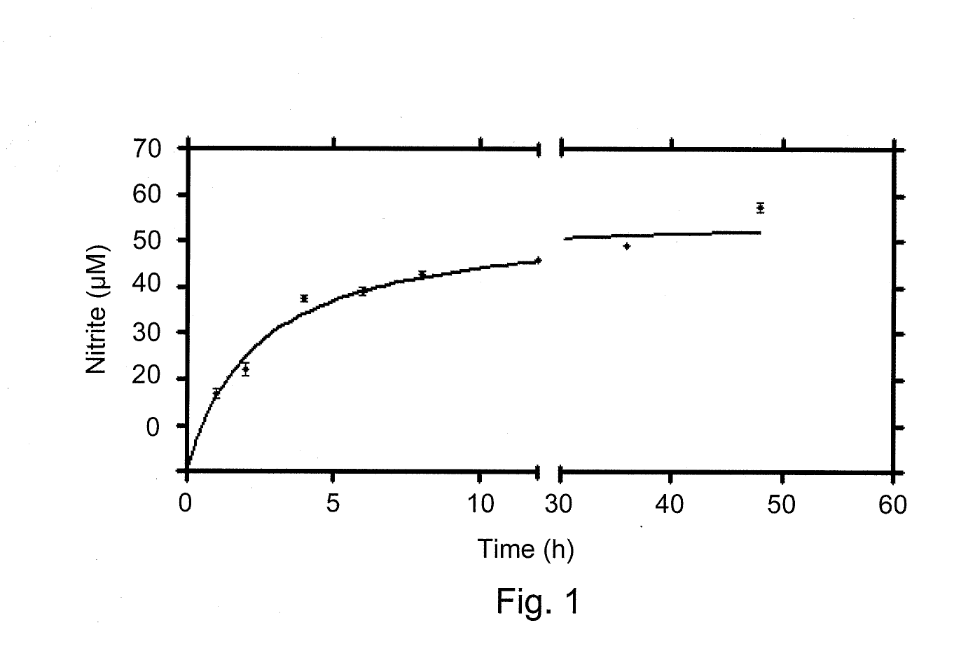

A 6-mm diameter Folysil® (Folysil is a trademark of Colopast A/S Corp., Holtedam 1 Humlebaek, Denmark) Foley catheter (cat. no. AA6118, Colopast Corp. Minneapolis Minn., USA) was cut aseptically into 3-cm sections. The cut sections were placed in a modified-exposure Petri dish which was then attached to a gNO cyclone delivering 20,000 ppm of gNO at a flow rate of 30 cc min⁻¹ for a period of 24 hours. After completion of the 24-h NO impregnation period, the gNO-treated catheter sections were aseptically transferred to sterile vials each containing 5 mL of sterile water (4 catheter sections per vial). A 100-μL aliquot was aseptically removed from each vial at the following time periods: 1, 2, 4, 6, 8, 12, 24, 36, 48 h, and the nitrites and nitrates contents were determined with the Griess test using a Griess reagent comprising 0.2% naphthylenediamine dihydrochloride, and 2% sulphanilamide in 5% phosphoric acid. FIG. 1 shows that in the 48 hours following the impregnation, NO was slowly released from the catheter sections. After being immersed in water for 24 hours (following the treatment), about 46 μM of nitrites had accumulated per 1 cm of catheter section per mL of water in the vials which equates to the release of about 1.4 ppm NO.

### Example 2

A 6-mm diameter Folysil® Foley catheter was cut aseptically into 3-cm sections. The cut sections were placed in a modified-exposure Petri dish which was then attached to a gNO cyclone delivering 20,000 ppm of gNO at a flow rate of 30 cc min⁻¹ for a period of 24 hours.

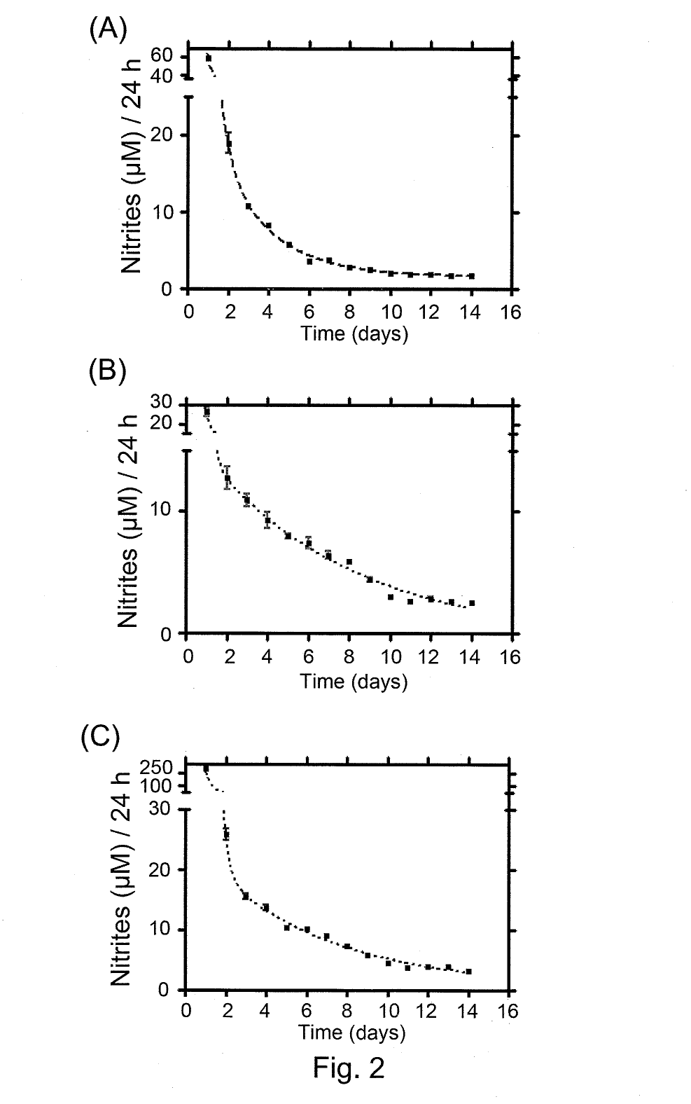

After the 24-h gNO impregnation period was complete, a first group of sections were aseptically transferred to sterile vials each containing 5 mL of sterile water (4 catheter sections per vial). A 100-μL aliquot was aseptically removed from each vial at daily intervals during the subsequent 14-day period, and the nitrites and nitrates contents in each aliquot were determined with the Griess test using the Griess reagent. FIG. 2(A) shows the release of NO gas from the catheter sections over the 14-day period after their immersion in water.

A second group of gNO-impregnated catheter sections were stored in a sterile air environment for a 7-day period after completion of the 24-h gNO impregnation period. The catheter sections were then aseptically transferred to sterile vials each containing 5 mL of sterile water (4 catheter sections per vial). A 100-μL aliquot was aseptically removed from each vial at daily intervals during the subsequent 14-day period, and the nitrites and nitrates contents were determined with the Griess test using the Griess reagent. FIG. 2(B) shows the release of NO gas from the 7-day-stored catheter sections over the 14-day period after their immersion in water.

A third group of gNO-impregnated catheter sections were stored in a sterile air environment for a 7-day period after the 24-h gNO impregnation period. The catheter sections were then aseptically transferred to sterile vials each containing 5 mL of sterilized urine (4 catheter sections per vial). The sterilized urine was replaced daily. The sterilized urine was produced by daily collection of fresh urine from a male volunteer, which was then filter-sterilized and supplemented with Ampicillin (50 μg mL⁻¹, final concentration). A 100-μL aliquot was aseptically removed from each vial each day prior to replacement of the sterile urine for the duration of the 14-day study. The nitrites and nitrates contents were determined with the Griess test using the Griess reagent. FIG. 2(C) shows the release of NO gas from the gNO-impregnated catheter sections over the 14-day period during their immersion in sterile urine.

### Example 3

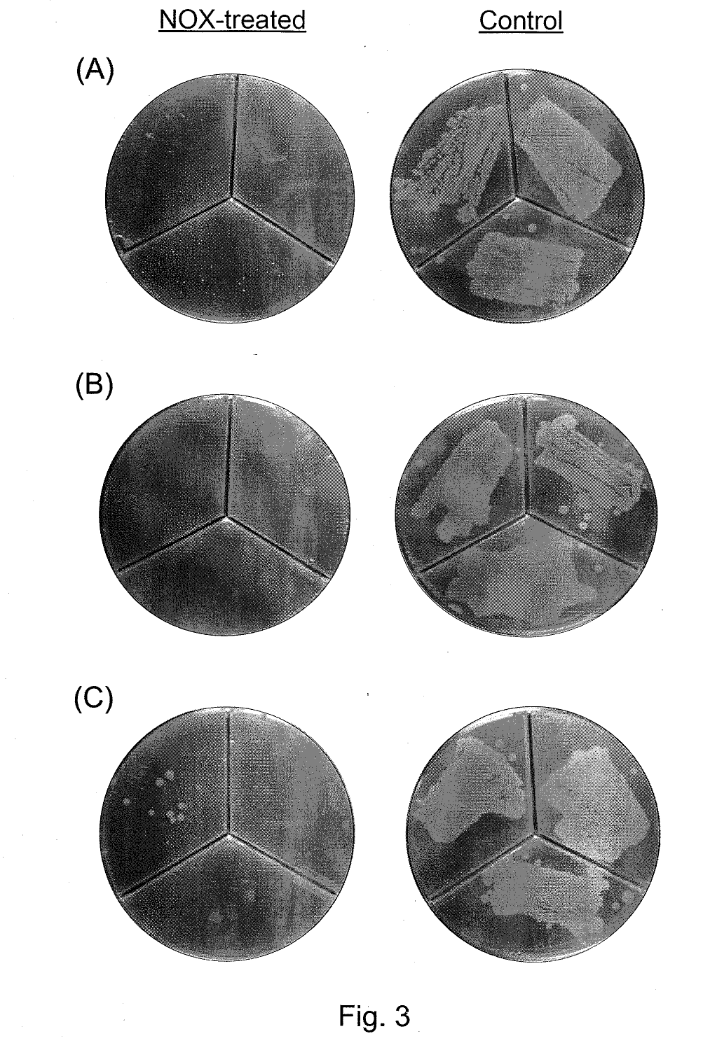

A 6-mm diameter Folysil® Foley catheter was cut aseptically into 3-cm sections. The cut sections were placed in a modified-exposure Petri dish which was then attached to a gNO cyclone delivering 20,000 ppm of gNO at a flow rate of 30 cc min⁻¹ for a period of 24 hours. “Air-stored” control sections of the cut Foley catheter sections were stored aseptically in airtight vials for 24 h. A subset of catheter sections from each treatment were immersed in one of three E. coli suspensions for 24 h at ambient room temperature. The three E. coli suspensions contained 10², 10³, and 10⁴ colony-forming units (CFU) mL⁻¹. At completion of the 24-h incubation period in the E. coli suspensions, the sections were removed and washed twice with sterile distilled water. The sections were then rolled across the surface of LB agar dispensed into 3-chambered Petri plates (one catheter section per chamber). The Petri plates were then incubated for 18 h at 37° C. FIG. 3(A) shows photographs comparing the growth of E. coli colonies in Petri plates on which gNO-impregnated catheter sections were rolled compared to Petri plates in which air-stored control catheter sections were rolled, wherein both sets of catheter sections had been immersed for 24 h in an E. coli suspension comprising 10² CFU mL⁻¹. FIG. 3(B) shows photographs comparing the growth of E. coli colonies in Petri plates on which gNO-impregnated catheter sections were rolled compared to Petri plates in which air-stored control catheter sections were rolled, wherein both sets of catheter sections had been immersed for 24 h in an E. coli suspension comprising 10³ CFU mL⁻¹. FIG. 3(C) shows photographs comparing the growth of E. coli colonies in Petri plates on which gNO-impregnated catheter sections were rolled compared to Petri plates in which air-stored control catheter sections were rolled, wherein both sets of catheter sections had been immersed for 24 h in an E. coli suspension comprising 10⁴ CFU mL⁻¹. FIGS. 3(A)-3(C) show that E. coli had established growth on the surfaces of the control catheter sections during the 24-h immersion period. FIG. 3(A) and 3(B) show that E. coli was not present on the surfaces of gNO-impregnated catheter sections that had been immersed for 24 h in E. coli suspensions comprising 10² CFU mL⁻¹ and 10³ CFU mL⁻¹ respectively. FIG. 3(C) shows that although some E. coli CFU were present on the surfaces of gNO-impregnated catheter sections immersed in an E. coli suspension comprising 10⁴ CFU mL⁻¹, the colonization of the surfaces of these impregnated catheters was very much reduced in comparison to the controls.

### Example 4

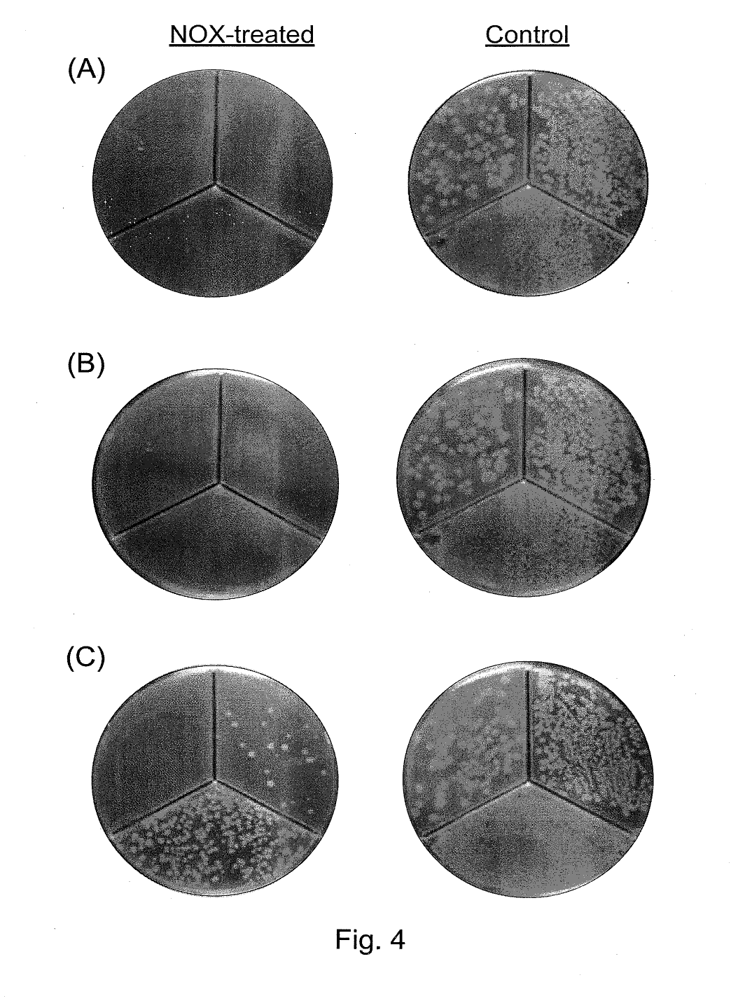

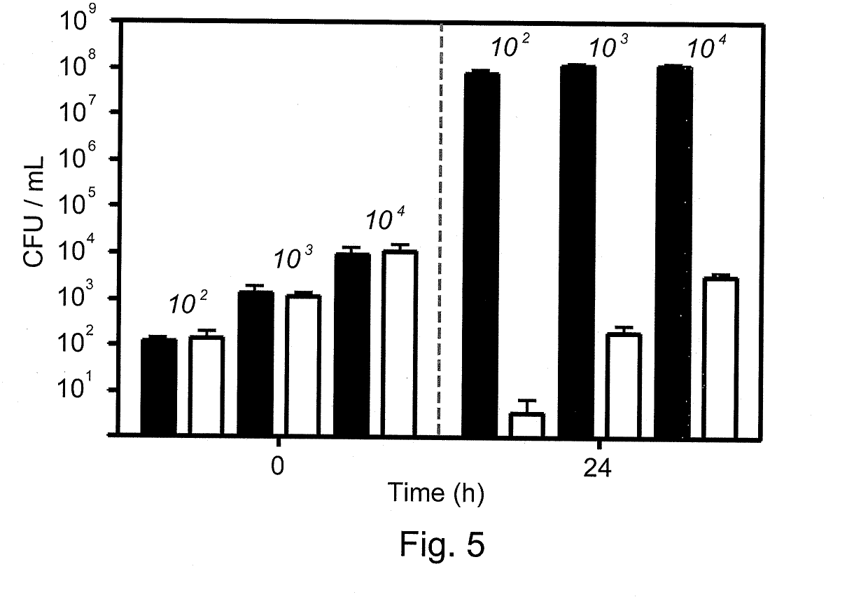

A 6-mm diameter Folysil® Foley catheter was cut aseptically into 3-cm sections. The cut sections were placed in a modified-exposure Petri dish which was then attached to a gNO cyclone delivering 20,000 ppm of gNO at a flow rate of 30 cc min⁻¹ for a period of 24 hours. “Air-stored” control sections of the cut Foley catheter sections were stored aseptically in airtight vials for 24 h. A subset of catheter sections from each treatment were immersed in one of three E. coli suspensions for 24 h at ambient room temperature. The three E. coli suspensions contained 10², 10³, and 10⁴ colony-forming units (CFU) mL⁻¹. At completion of the 24-h incubation period, a 100-μL aliquot was aseptically removed from each E. coli suspension and was plated onto a chamber in 3-chambered Petri plates containing LB agar. The Petri plates were then incubated for 18 h at 37° C. FIG. 4(A) compares photographs of the growth of E. coli colonies from E. coli suspensions comprising 10² CFU mL⁻¹ wherein gNO-impregnated catheter sections were incubated to suspensions wherein air-stored control catheter sections were incubated. FIG. 4(B) compares photographs of the growth of E. coli colonies from E. coli suspensions comprising 10³ CFU mL⁻¹ wherein gNO-impregnated catheter sections were incubated to suspensions wherein air-stored control catheter sections were incubated. FIG. 4(C) compares photographs of the growth of E. coli colonies from E. coli suspensions comprising 10⁴ CFU mL⁻¹ wherein gNO-impregnated catheter sections were incubated to suspensions wherein air-stored control catheter sections were incubated. FIGS. 4(A)-4(C) show that prolific growth of E. coli in the suspensions wherein control catheter sections had been immersed for 24 h. FIG. 4(A) and 4(B) show that no E coli CFU were isolated from the E. coli suspensions comprising 10² CFU mL⁻¹ and 10³ CFU mL⁻¹ respectively, wherein gNO-impregnated catheter sections had been immersed for 24 h. FIG. 4(C) shows that although some E. coli CFU were isolated from the E. coli suspensions comprising 10⁴ CFU mL⁻¹ wherein gNO-impregnated catheter sections were incubated for 24 h, the numbers of viable cells in the E. coli suspensions comprising 10⁴ CFU mL⁻¹, in comparison to the controls, were significantly reduced after the 24-h period of exposure to the gNO-impregnated catheter sections. FIG. 5 is a chart showing that at time 0, i.e., after immersion of control catheter sections followed by their immediate removal after which aliquots of the samples of the E. coli suspensions onto Petri plates containing LB agar, about the same numbers of E. coli CFU were isolated from each suspension, i.e., 10² CFU mL⁻¹, 10³ CFU mL⁻¹ and 10⁴ CFU mL⁻¹ respectively. After 24-h incubation with immersed control catheter sections, about 10⁸ CFU ml⁻¹ of E. coli were present in each of the E. coli suspensions. However, after 24-h of exposure to gNO-impregnated catheter sections, about 5 CFU ml⁻¹ of E. coli were present in the E. coli suspension having an initial concentration of 10² CFU ml⁻¹ (i.e., an 95% reduction), about 120 CFU ml⁻¹ of E. coli were present in the E. coli suspension having an initial concentration of 10³ CFU ml⁻¹ (i.e., an 82% reduction), and about 1,500 CFU ml⁻¹ of E. coli were present in the E. coli suspension having an initial concentration of 10⁵ CFU ml⁻¹ (i.e., an 85% reduction). The data from this example show that gNO-impregnated catheters according to this invention released sufficient amounts of NO to significantly reduce the numbers of viable E. coli cells in the E. coli suspensions, thereby demonstrating the antimicrobial properties of the gNO-impregnated catheters.

### Example 5

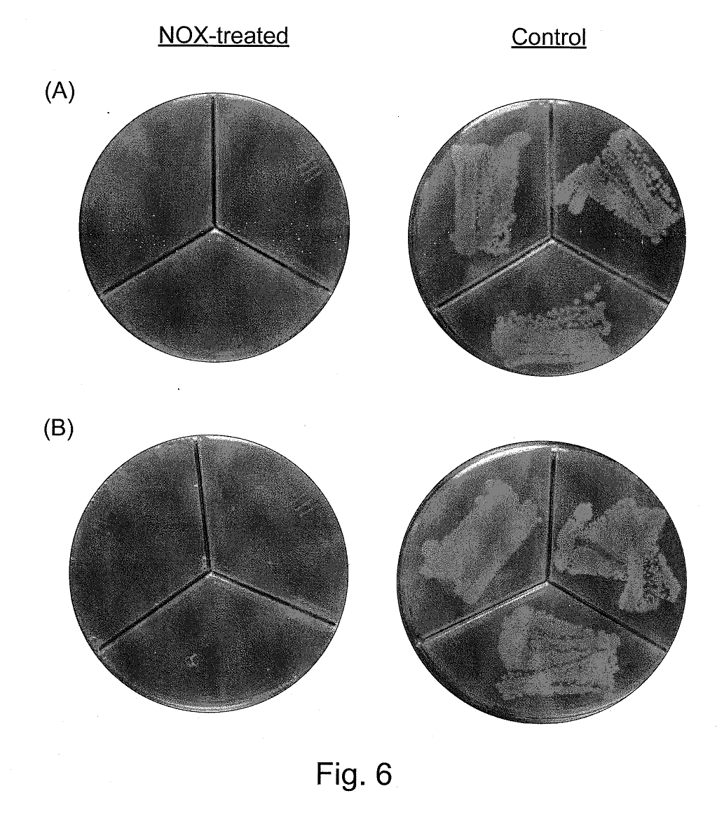

A 6-mm diameter Folysil® Foley catheter was cut aseptically into 3-cm sections. The cut sections were placed in a modified-exposure Petri dish which was then attached to a gNO cyclone delivering 20,000 ppm of gNO at a flow rate of 30 cc min⁻¹ for a period of 24 hours. “Air-stored” control sections of the cut Foley catheter sections were stored aseptically in airtight vials for 24 h. Half of the gNO-impregnated catheter sections were stored aseptically in airtight vials for 7 days, while the other half were stored for 7 days in sterile vials filled with sterile water. Half of the control catheter sections were stored aseptically in airtight vials for 7 days, while the other half were stored for 7 days in sterile vials filled with sterile water. At the end of the 7-day storage period, a subset of catheter sections from each treatment was immersed in a 10³ E. coli suspension for 1 min at ambient room temperature after which, catheter sections were aseptically transferred to test tubes containing PBS (1 catheter section per tube). After a 24-h incubation period in the PBS, the sections were removed and washed twice with sterile distilled water. The sections were then rolled across the surface of LB agar dispensed into 3-chambered Petri plates (one catheter section per chamber). The Petri plates were then incubated for 18 h at 37° C. FIG. 6(A) shows photographs comparing the growth of E. coli colonies in Petri plates on which 7-day air-stored gNO-impregnated catheter sections were rolled compared to Petri plates in which 7-day air-stored control catheter sections were rolled, after both sets of catheter sections had been immersed for 1 min in an E. coli suspension comprising 10³ CFU mL⁻¹, and then incubated for 24 h in PBS. FIG. 6(B) shows photographs comparing the growth of E. coli colonies in Petri plates on which 7-day sterile-water-stored gNO-impregnated catheter sections were rolled compared to Petri plates in which 7-day sterile-water-stored control catheter sections were rolled, after both sets of catheter sections had been immersed for 1 min in an E. coli suspension comprising 10³ CFU mL⁻¹, and then incubated for 24 h in PBS. These photographs confirm the stability of the gNO-impregnated catheter sections after storage for 7 days in sterile-air and sterile-water environments.

### Example 6

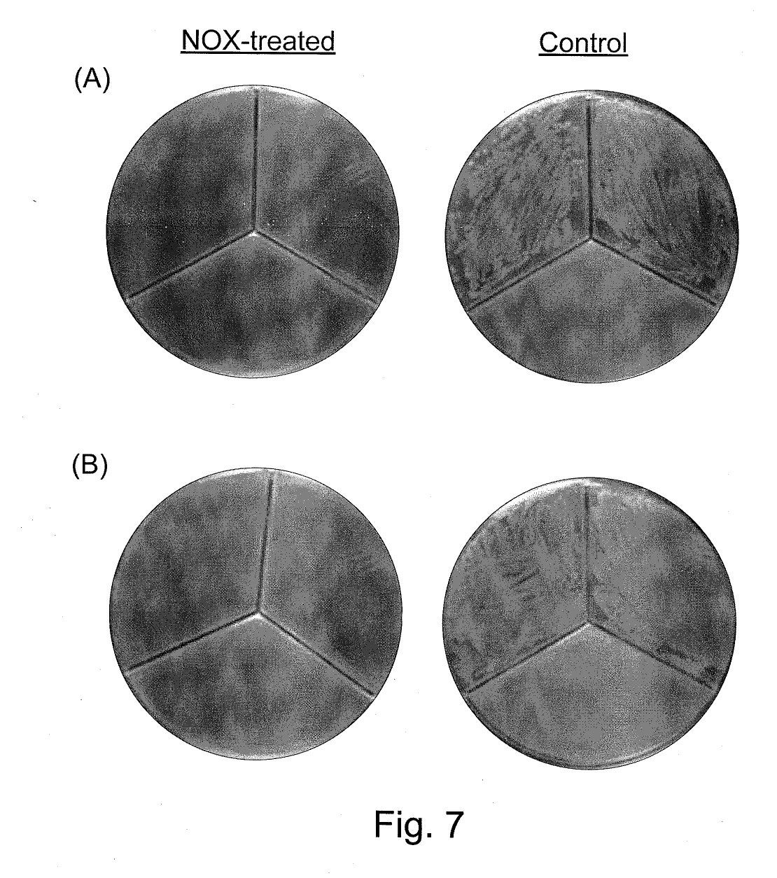

A 6-mm diameter Folysil® Foley catheter was cut aseptically into 3-cm sections. The cut sections were placed in a modified-exposure Petri dish which was then attached to a gNO cyclone delivering 20,000 ppm of gNO at a flow rate of 30 cc min⁻¹ for a period of 24 hours. “Air-stored” control sections of the cut Foley catheter sections were stored aseptically in airtight vials for 24 h. Half of the gNO-impregnated catheter sections were stored aseptically in airtight vials for 7 days, while the other half were stored for 7 days in sterile vials filled with sterile water. Half of the control catheter sections were stored aseptically in airtight vials for 7 days, while the other half were stored for 7 days in sterile vials filled with sterile water. At the end of the 7-day storage period, a subset of catheter sections from each treatment was immersed in a 10³ E. coli suspension for 1 min at ambient room temperature after which, catheter sections were aseptically transferred to test tubes containing PBS (1 catheter section per tube). At the end of the 7-day storage period, a subset of catheter sections from each treatment was immersed in a 10³ E. coli suspension for 1 min at ambient room temperature after which, catheter sections were aseptically transferred to test tubes containing PBS (1 catheter section per tube). After a 24-h incubation period in the PBS, the sections were removed and washed twice with sterile distilled water. At the end of the 24-h incubation period, a subsample of PBS were taken from each of 3 PBS tubes and plated onto a chamber in a 3-chambered Petri plate containing B agar. FIG. 7(A) compares photographs of the growth of E. coli colonies from PBS wherein 7-day air-stored gNO-impregnated catheter sections were incubated to suspensions wherein 7-day air-stored control catheter sections were incubated. FIG. 7(B) compares photographs of the growth of E. coli colonies from PBS wherein 7-day sterile-water-stored gNO-impregnated catheter sections were incubated to suspensions wherein 7-day sterile-water-stored control catheter sections were incubated. These photographs demonstrate the release of antimicrobial amounts of NO gas from gNO-impregnated catheter sections after storage for 7 days in sterile-air and sterile-water environments.

### Example 7

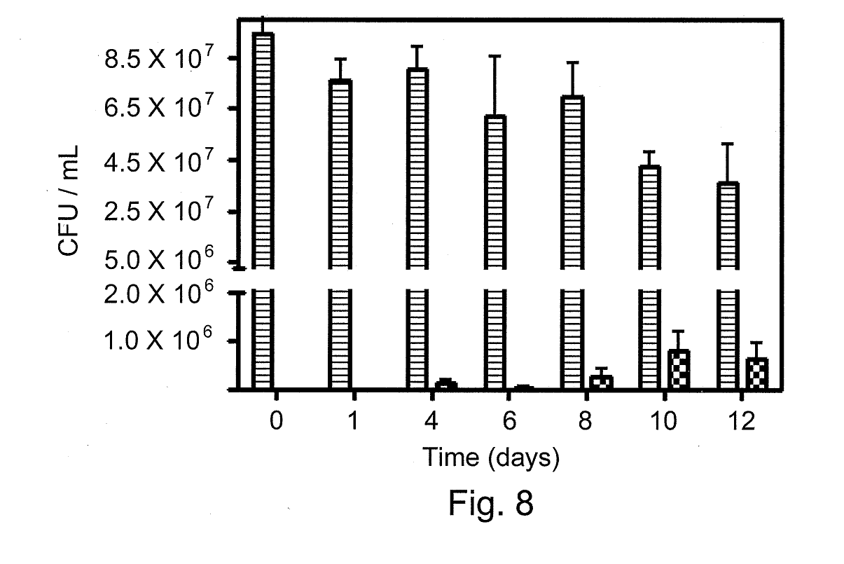

A 6-mm diameter Folysil® Foley catheter was cut aseptically into 3-cm sections. The cut sections were placed in a modified-exposure Petri dish which was then attached to a gNO cyclone delivering 20,000 ppm of gNO at a flow rate of 30 cc min⁻¹ for a period of 24 hours. “Air-stored” control sections of the cut Foley catheter sections were stored aseptically in airtight vials for 24 h. The control catheter sections and the gNO-impregnated catheter sections were separately stored aseptically in airtight vials for 12 days. At the end of the 12-day storage period, a subset of catheter sections from each treatment was immersed in a 10³ E. coli suspension for 1 min at ambient room temperature after which, catheter sections were aseptically transferred to test tubes containing PBS (1 catheter section per tube). After a 24-h incubation period in the PBS, aliquots from the “control” PBS tubes and from the “NO-impregnated” PBS tubes were plated Petri plates containing LB agar. FIG. 8 shows that: (a) E. coli proliferated in PBS wherein E. coli-dipped control catheter sections had been incubated for 24 h, and (b) NO released from NO-impregnated catheter sections that had been stored for up to 12 days prior to dipping in E. coli suspension, prior to incubation for 24 h in PBS, significantly reduced or eliminated E. coli proliferation in PBS, thus demonstrating the antimicrobial and self-sterilizing properties of the examplary NO-impregnated catheters of the present invention.

It is also within the scope of the present invention to infiltrate the mounting plates of ostomy pouching systems with nitric oxide using the methods described herein for providing antimicrobial gas-releasing properties to the mounting plates to prevent biofilms from forming thereon when installed onto a stoma of an individual who has undergone a colostomy procedure, and for the prevention of microbial infections of the individual's stoma area.

Those skilled in these arts will understand that the gNO-impregnated medical tubings and/or gNO-infiltrated medical appliances of the present invention are storable for extended periods of time in suitable sterile air environments such as exemplified by aseptic moisture impermeable packagings. After such gNO-impregnated medical tubings and/or gNO-infiltrated medical appliances are communicatingly installed thereinto a patient and upon contact with moisture, the medical tubings and/or the medical appliances will release NO gas thereabout for an extended period of time thereby inhibiting the formation of biofilms on the outer surfaces and inner surfaces of the tubings and/or appliances. Furthermore, the released NO gas will inhibit microbial growth and proliferation in the immediate environments about the installed medical tubings and medical devices.

1. A self-sterilizing biofilm-inhibiting nitric oxide-releasing medical appliance for installation into a patient, said medical appliance configured for conveying materials therethrough, the medical appliance comprising:
a gas-permeable cured resin material selected from the group consisting of curable silicones, polyvinyl acetates, thermoplastic elastomers, acrylonitrile-butadiene-styrene copolymer rubber, polyurethanes and selected combinations thereof, said gas-permeable cured resin material comprising a matrix suitable for releasably sequestering therein permeating gases; and
a plurality of nitric-oxide-releasing moieties sequestered therein said gas-permeable resin material;
wherein said nitric oxide is released from at least a portion of said plurality of nitric oxide releasing moieties upon contact of the medical appliance with a moisture source.

2. The medical appliance of claim 1, wherein the medical appliance comprises a medical tubing selected from the group consisting of endotracheal tubes, crichotracheotomy tubes, urinary catheters, wound drainage tubes, central intravenous catheters, and peripheral intravenous catheters.

3. The medical appliance of claim 2, wherein the medical appliance additionally comprises at least one component cooperable with said medical tubing.

4. The medical appliance of claim 1, wherein said medical appliance comprises a pouching system.

5. The medical appliance of claim 4, wherein said medical appliance comprises a mounting plate component of the pouching system.

6. The medical appliance of claim 5, wherein the mounting plate component comprises an adhesive patch.

7. The medical appliance of claim 1, wherein the moisture source is one of a bodily fluid produced by the patient, a moisture-containing material produced by the patient, a therapeutic solution, and an irrigant.

8. The medical appliance of claim 1, wherein the gas-permeable cured resin material comprises at least one nitric oxide-sequestering chelating agent selected from the group consisting of sodium nitrite, nitrosothiols, dipyridoxyl chelating agents, L-arginine, organic nitrates, organic nitrites, thionitrates, thionitrites, N-oxo-N-nitrosamines, N-nitrosamines, sydnonimines, 2-hydroxyimino-5-nitro-alkenamides, diazenium diolates, oxatriazolium compounds, oximes, syndomines, molsidomine and derivatives thereof, pirsidomine, furoxanes, nitrosonium salts, and combinations thereof.

9. The medical appliance of claim 1, additionally coated with a chemical composition configured to react with a moisture source thereby causing release of nitric oxide from at least a portion of the nitric-oxide-releasing moieties sequestered therein said gas-permeable resin material.

10. A process for producing a biofilm-inhibiting nitric oxide-releasing antimicrobial medical appliance for installation into a patient's body, said medical appliance configured for conveying materials therethrough and additionally configured for releasing nitric oxide therefrom when said medical appliance is contacted by a moisture source, the process comprising:
placing into a sealable chamber, a plurality of medical appliances comprising a gas-permeable cured resin material selected from the group consisting of curable silicones, polyvinyl acetates, thermoplastic elastomers, acrylonitrile-butadiene-styrene copolymer rubber, polyurethanes and selected combinations thereof, said gas-permeable cured resin material configured for releasably sequestering therein permeating gases;
controllably saturating said chamber with a plurality of selected nitric oxide-releasing moieties wherein the plurality of nitric oxide-releasing moieties permeates said gas-permeable cured resin material whereby at least a portion of the plurality of nitric oxide-releasing moieties is releasably sequestered within and throughout the gas-permeable cured resin material; and
packaging said medical appliance permeated with nitric oxide-releasing moieties within a gas-impermeable packaging.

11. The process of claim 10, wherein the gas-permeable cured resin material comprises at least one nitric oxide-sequestering chelating agent selected from the group consisting of nitrite, nitrosothiols, dipyridoxyl chelating agents, L-arginine, organic nitrates, organic nitrites, thionitrates, thionitrites, N-oxo-N-nitrosamines, N-nitrosamines, sydnonimines, 2-hydroxyimino-5-nitro-alkenamides, diazenium diolates, oxatriazolium compounds, oximes, syndomines, molsidomine and derivatives thereof, pirsidomine, furoxanes, nitrosonium salts, and combinations thereof.

12. The process of claim 10, wherein the medical appliance is additionally coated with a chemical composition configured to react with a moisture source whereby said reaction causes release of nitric oxide from at least a portion of the nitric-oxide-releasing moieties sequestered therein said gas-permeable resin material.

13. The process of claim 10, wherein the moisture source is a bodily fluid produced by the patient, a moisture-containing material produced by the patient, a therapeutic solution, or an irrigant.

14. The process of claim 10, wherein the medical appliance comprises a medical tubing selected from the group consisting of endotracheal tubes, crichotracheotomy tubes, urinary catheters, wound drainage tubes, central intravenous catheters, and peripheral intravenous catheters.

15. The process of claim 10, wherein the medical appliance additionally comprises at least one component cooperable with said medical tubing.

16. The process of claim 10, wherein the medical appliance comprises a pouching system.

17. The process of claim 16, wherein the medical appliance comprises a mounting plate component of a pouching system.

18. The process of claim 17, wherein the mounting plate component comprises an adhesive patch.

19. The process of claim 10, wherein the chamber is controllably saturated with gaseous nitric oxide at a concentration selected from the range of about 5 ppm to about 1,000,000 ppm at a flow rate selected from the range of about 1 cc mL⁻¹ to about 200 cc mL⁻¹ for a period of time selected from the range of about 15 min to about 120 h.

20. The process of claim 10, wherein the chamber is controllably saturated with gaseous nitric oxide at a concentration selected from the range of about 500 ppm to about 100,000 ppm at a flow rate selected from the range of about 5 cc mL⁻¹ to about 100 cc mL⁻¹ for a period of time selected from the range of about 30 min to about 48 h.

21. The process of claim 10, wherein the chamber is controllably saturated with gaseous nitric oxide at a concentration selected from the range of about 5,000 ppm to about 50,000 ppm at a flow rate selected from the range of about 10 cc mL⁻¹ to about 75 cc mL⁻¹ for a period of time selected from the range of about 1 h to about 48 h.

22. The process of claim 10, wherein the chamber is controllably saturated with gaseous nitric oxide at a concentration selected from the range of about 10,000 ppm to about 35,000 ppm at a flow rate selected from the range of about 15 cc mL⁻¹ to about 40 cc mL⁻¹ for a period of time selected from the range of about 6 h to about 36 h.

23. The process of claim 10, wherein the chamber is controllably saturated with gaseous nitric oxide at a concentration selected from the range of about 15,000 ppm to about 30,000 ppm at a flow rate selected from the range of about 20 cc mL⁻¹ to about 35 cc mL⁻¹ for a period of time selected from the range of about 12 h to about 30 h.

24. The process of claim 10, wherein the chamber is controllably saturated with gaseous nitric oxide at a concentration selected from the range of about 18,000 ppm to about 25,000 ppm at a flow rate selected from the range of about 20 cc mL⁻¹ to about 30 cc mL⁻¹ for a period of time selected from the range of about 18 h to about 30 h.

25. The process of claim 10, wherein the chamber is controllably saturated with gaseous nitric oxide at a concentration of about 20,000 ppm at a flow rate of about 20 cc mL⁻¹ for a period of time of about 24 h.
